(19) United States
(12) Patent Application Publication
(10) Pub. No.: US 2024/0260929 A1
(43) Pub. Date: Aug. 8, 2024

(54) MEDICAL IMAGE DIAGNOSTIC SYSTEM AND METHOD OF CONTROLLING MEDICAL IMAGE DIAGNOSTIC SYSTEM

(71) Applicant: FUJIFILM Corporation, Tokyo (JP)

(72) Inventors: Hidetoshi TAKAYAMA, Kanagawa (JP); Keiji TSUBOTA, Kanagawa (JP)

(73) Assignee: FUJIFILM Corporation, Tokyo (JP)

(21) Appl. No.: 18/422,138

(22) Filed: Jan. 25, 2024

(30) Foreign Application Priority Data
Feb. 8, 2023 (JP) 2023-017342

(51) Int. Cl. A61B 8/08; A61B 8/00; G06T 3/40; G06T 11/60

(52) U.S. Cl. CPC A61B 8/0825; A61B 8/403; A61B 8/461; G06T 3/40; G06T 11/60; G06T 2210/36; G06T 2210/41

(57) ABSTRACT

Provided are a medical image diagnostic system that allows a user to easily and accurately examine a mammary gland region of a subject while preventing the subject from confirming a detailed shape of the mammary gland region, and a method of controlling the medical image diagnostic system.

The medical image diagnostic system includes: a compression plate that compresses a breast of a subject; an ultrasound probe; a mammary gland region detection unit that detects a mammary gland region in a radiation image of the breast compressed by the compression plate; a low-resolution image generation unit that generates a low-resolution image obtained by reducing a resolution of the mammary gland region detected by the mammary gland region detection unit; and an image drawing unit that draws the low-resolution image generated by the low-resolution image generation unit on the compression plate, in which the mammary gland region of the breast compressed by the compression plate is scanned with the ultrasound probe based on the low-resolution image drawn on the compression plate.

### CROSS-REFERENCE TO RELATED APPLICATIONS

The present application claims priority under 35 U.S.C. § 119 to Japanese Patent Application No. 2023-017342, filed on Feb. 8, 2023. The above application is hereby expressly incorporated by reference, in its entirety, into the present application.

### BACKGROUND OF THE INVENTION

### 1. Field of the Invention

The present invention relates to a medical image diagnostic system that examines a breast of a subject and a method of controlling the medical image diagnostic system.

### 2. Description of the Related Art

In the related art, a breast of a subject has been examined by capturing a radiation image using a so-called radiography apparatus and capturing an ultrasound image using a so-called ultrasound diagnostic apparatus. For example, JP2020-127650A discloses a medical image diagnostic system capable of capturing a radiation image in a state where a breast of a subject is compressed by a compression plate and subsequently capturing an ultrasound image in a state where the breast is compressed. The medical image diagnostic system disclosed in JP2020-127650A projects a mammary gland region of the breast onto the compression plate that is compressing the breast of the subject by irradiating the compression plate with visible light so that a user such as a doctor or a technician can easily examine the mammary gland region.

### SUMMARY OF THE INVENTION

In the technique of JP2020-127650A, a detailed shape of the mammary gland region is projected onto the compression plate, so that the subject under an examination may unintentionally confirm the projected mammary gland region. Some subjects may avoid confirming the detailed shape of their own mammary gland region.

The present invention has been made in order to solve such a problem in the related art, and an object of the present invention is to provide a medical image diagnostic system that allows a user to easily and accurately examine a mammary gland region of a subject while preventing the subject from confirming a detailed shape of the mammary gland region, and a method of controlling the medical image diagnostic system.

According to the following configuration, the above-described object can be achieved.
[1] A medical image diagnostic system comprising: a compression plate that compresses a breast of a subject; an ultrasound probe; a mammary gland region detection unit that detects a mammary gland region in a radiation image of the breast compressed by the compression plate; a low-resolution image generation unit that generates a low-resolution image obtained by reducing a resolution of the mammary gland region detected by the mammary gland region detection unit; and an image drawing unit that draws the low-resolution image generated by the low-resolution image generation unit on the compression plate, in which the mammary gland region of the breast compressed by the compression plate is scanned with the ultrasound probe based on the low-resolution image drawn on the compression plate.
[2] The medical image diagnostic system according to [1], further comprising: a probe-disposed image recognition unit that recognizes the low-resolution image that is drawn on the compression plate by the image drawing unit and on which a distal end of the ultrasound probe is disposed.
[3] The medical image diagnostic system according to [2], in which the image drawing unit changes a display aspect on the compression plate of the low-resolution image recognized by the probe-disposed image recognition unit.
[4] The medical image diagnostic system according to [2] or [3], in which the mammary gland region detection unit detects a plurality of the mammary gland regions, the low-resolution image generation unit generates a plurality of the low-resolution images obtained by reducing resolutions of the plurality of mammary gland regions, the image drawing unit draws the plurality of low-resolution images on the compression plate, and the medical image diagnostic system further comprises a proportion notification unit that calculates a proportion of the low-resolution image recognized by the probe-disposed image recognition unit from a start of an examination using the ultrasound probe to the present, to the plurality of low-resolution images drawn on the compression plate, and that notifies of the proportion.
[5] The medical image diagnostic system according to any one of [1] to [4], further comprising: a mammary gland position display image generation unit that generates a mammary gland position display image indicating a position of the mammary gland region based on the mammary gland region detected by the mammary gland region detection unit, in which the image drawing unit draws the mammary gland position display image generated by the mammary gland position display image generation unit to be superimposed on the low-resolution image drawn on the compression plate.
[6] The medical image diagnostic system according to any one of [1] to [5], in which the image drawing unit performs drawing by a projection mapping device that draws the low-resolution image on the compression plate by projection mapping.
[7] The medical image diagnostic system according to any one of [1] to [5], in which the image drawing unit performs drawing by a transparent display device disposed on the compression plate.
[8] A method of controlling a medical image diagnostic system, the method comprising: detecting a mammary gland region in a radiation image of a breast of a subject compressed by a compression plate; generating a low-resolution image obtained by reducing a resolution of the detected mammary gland region; and drawing the generated low-resolution image on the compression plate, in which the mammary gland region of the breast compressed by the compression plate is scanned with an ultrasound probe based on the low-resolution image drawn on the compression plate.

According to the present invention, the medical image diagnostic system comprises: a compression plate that compresses a breast of a subject; an ultrasound probe; a mammary gland region detection unit that detects a mammary gland region in a radiation image of the breast compressed by the compression plate; a low-resolution image generation unit that generates a low-resolution image obtained by reducing a resolution of the mammary gland region detected by the mammary gland region detection unit; and an image drawing unit that draws the low-resolution image generated by the low-resolution image generation unit on the compression plate, in which the mammary gland region of the breast compressed by the compression plate is scanned with the ultrasound probe based on the low-resolution image drawn on the compression plate. Therefore, it is possible for a user to easily and accurately examine the mammary gland region of the subject while preventing the subject from confirming a detailed shape of the mammary gland region.

### BRIEF DESCRIPTION OF THE DRAWINGS

FIG. 1 is a block diagram showing a configuration of a medical image diagnostic system according to Embodiment 1 of the present invention.

FIG. 2 is a block diagram showing a configuration of a radiography apparatus in Embodiment 1 of the present invention.

FIG. 3 is a diagram schematically showing an example of an exterior of the radiography apparatus in Embodiment 1 of the present invention.

FIG. 4 is a diagram schematically showing an example of a mammary gland region in a radiation image of a breast of a subject.

FIG. 5 is a diagram schematically showing an example of a low-resolution image.

FIG. 6 is a diagram schematically showing another example of the low-resolution image.

FIG. 7 is a block diagram showing a configuration of a console in Embodiment 1 of the present invention.

FIG. 8 is a block diagram showing a configuration of an ultrasound diagnostic apparatus in Embodiment 1 of the present invention.

FIG. 9 is a block diagram showing a configuration of a transmission/reception circuit in Embodiment 1 of the present invention.

FIG. 10 is a block diagram showing a configuration of an image generation unit in Embodiment 1 of the present invention.

FIG. 11 is a flowchart showing an operation of the medical image diagnostic system according to Embodiment 1 of the present invention.

FIG. 12 is a block diagram showing a configuration of a radiography apparatus in a modification example of Embodiment 1 of the present invention.

FIG. 13 is a block diagram showing a configuration of a radiography apparatus in Embodiment 2 of the present invention.

FIG. 14 is a diagram schematically showing an example of a mammary gland position display image.

FIG. 15 is a block diagram showing a configuration of a radiography apparatus in Embodiment 3 of the present invention.

FIG. 16 is a diagram schematically showing an example of a low-resolution image on which a distal end of an ultrasound probe is disposed.

FIG. 17 is a block diagram showing a configuration of an ultrasound diagnostic apparatus in a modification example of Embodiment 3 of the present invention.

FIG. 18 is a block diagram showing a configuration of a radiography apparatus in the modification example of Embodiment 3 of the present invention.

FIG. 19 is a block diagram showing a configuration of a radiography apparatus in Embodiment 4 of the present invention.

FIG. 20 is a diagram showing an example of notification of a proportion of a recognized low-resolution image.

### DESCRIPTION OF THE PREFERRED EMBODIMENTS

Hereinafter, embodiments of the present invention will be described with reference to the accompanying drawings.

Description of configuration requirements described below may be made based on a typical embodiment of the present invention, but the present invention is not limited to such an embodiment.

In the present specification, the numerical range represented by “to” means a range including numerical values denoted before and after “to” as a lower limit value and an upper limit value.

In the present specification, the terms “same” and “identical” include an error range generally allowed in the technical field.

### Embodiment 1

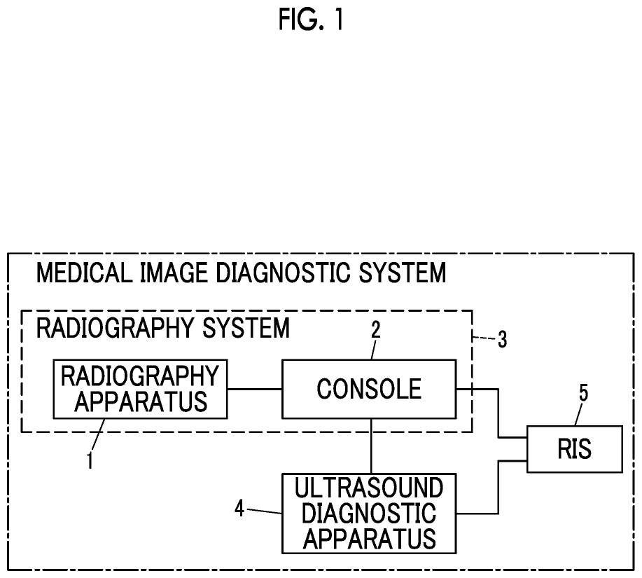

FIG. 1 shows a configuration of a medical image diagnostic system according to Embodiment 1 of the present invention. The medical image diagnostic system comprises a radiography system 3 including a radiography apparatus 1 and a console 2, an ultrasound diagnostic apparatus 4 connected to the console 2, and a radiology information system (RIS) 5 connected to the console 2 and the ultrasound diagnostic apparatus 4.

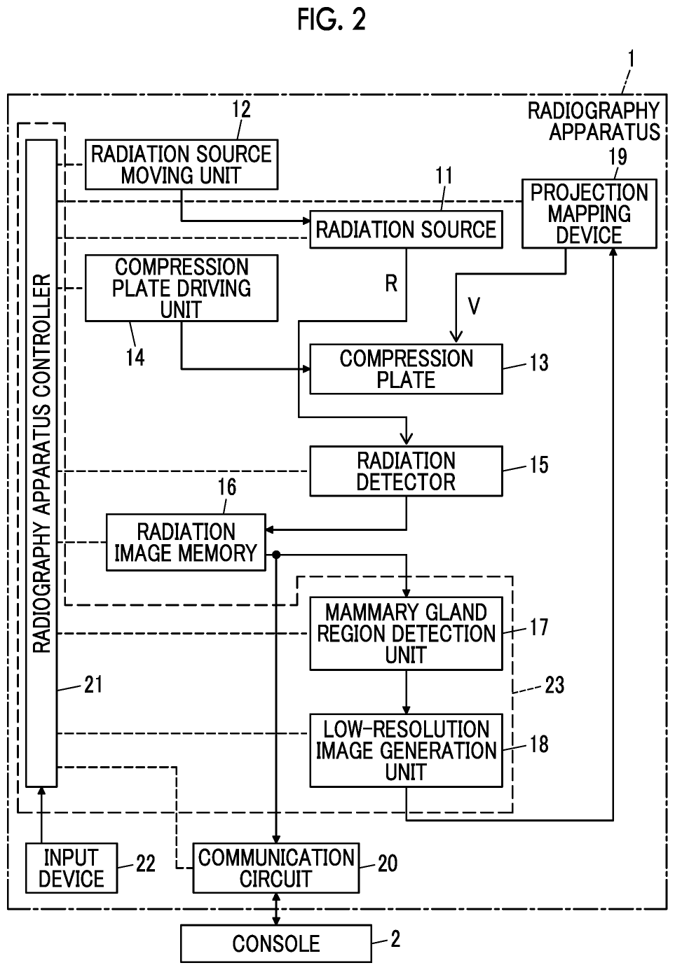

In FIG. 2, the radiography apparatus 1 includes a radiation source 11 and a radiation source moving unit 12 connected to the radiation source 11. In addition, the radiography apparatus 1 includes a compression plate 13 and a compression plate driving unit 14 connected to the compression plate 13. In addition, the radiography apparatus 1 includes a radiation detector 15. A radiation image memory 16 is connected to the radiation detector 15. A mammary gland region detection unit 17 and a low-resolution image generation unit 18 are sequentially connected to the radiation image memory 16. A projection mapping device 19 is connected to the low-resolution image generation unit 18. In addition, a communication circuit 20 is connected to the radiation image memory 16. The communication circuit 20 is connected to the console 2.

In addition, a radiography apparatus controller 21 is connected to the radiation source 11, the radiation source moving unit 12, the compression plate driving unit 14, the radiation detector 15, the radiation image memory 16, the mammary gland region detection unit 17, the low-resolution image generation unit 18, the projection mapping device 19, and the communication circuit 20. An input device 22 is connected to the radiography apparatus controller 21. In addition, the mammary gland region detection unit 17, the low-resolution image generation unit 18, and the radiography apparatus controller 21 constitute a processor 23 for the radiography apparatus 1. In addition, an image drawing unit is formed by the projection mapping device 19 and the radiography apparatus controller 21.

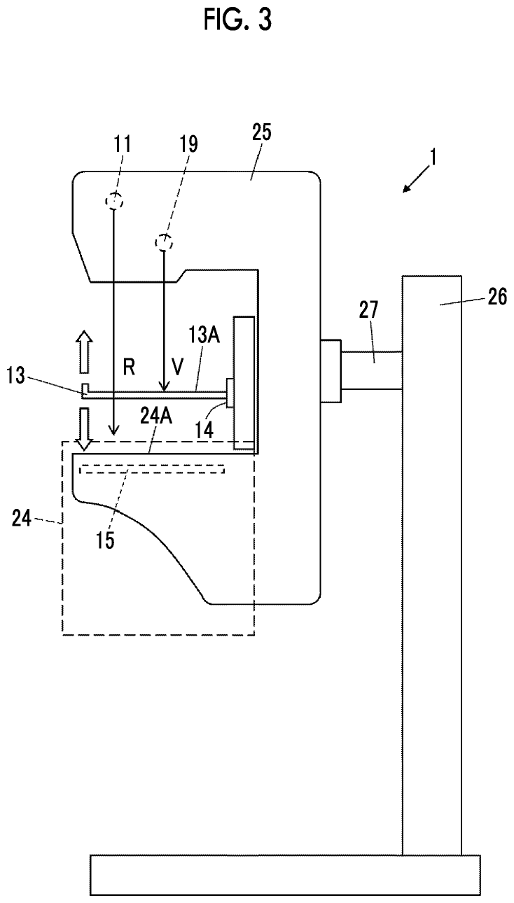

In addition, as shown in FIG. 3, the radiography apparatus 1 comprises an imaging table 24 located directly below the compression plate 13. An imaging surface 24A on which a breast of a subject is disposed during an examination is formed on the imaging table 24. In addition, the radiation detector 15 is built in the imaging table 24. The imaging table 24 is made of, for example, a carbon material, and transmits radiation R emitted from the radiation source 11. The radiation R transmitted through the imaging table 24 reaches the radiation detector 15.

The radiography apparatus 1 has an arm part 25, a base 26, and a shaft part 27. The arm part 25 is movably held in an up-down direction (height direction) by the base 26. The shaft part 27 connects the arm part 25 to the base 26. The arm part 25 can be rotated with respect to the base 26 with the shaft part 27 as a rotation shaft.

The radiation source 11 emits the radiation R by applying a tube voltage under the control of the radiography apparatus controller 21.

The radiation source moving unit 12 changes an inclination angle of the radiation source 11 and adjusts an irradiation direction of the radiation R emitted from the radiation source 11 by rotating the arm part 25 with respect to the base 26 with the shaft part 27 as a rotation shaft under the control of the radiography apparatus controller 21.

The compression plate driving unit 14 moves the compression plate 13 in the up-down direction under the control of the radiography apparatus controller 21.

The compression plate 13 compresses the breast of the subject by interposing the breast of the subject disposed on the imaging surface 24A of the imaging table 24 between the compression plate 13 and the imaging table 24. The compression plate 13 is preferably transparent in order to perform alignment during the compression of the breast and confirmation of a compression state of the breast, and is formed of a material having excellent transmittance to the radiation R and an ultrasonic wave. As a material of the compression plate 13, for example, a resin material such as polymethylpentene (PMP), polycarbonate (PC), acryl, polypropylene (PP), and polyethylene terephthalate (PET) can be used. In particular, polymethylpentene is suitable as the material of the compression plate 13 because it has low rigidity, excellent elasticity, and flexibility, and its acoustic impedance, which affects a reflectivity of the ultrasonic wave, is close to that of a human body (breast).

In a case in which a radiation image is captured, the radiation R is emitted from the radiation source 11 in a state where the breast of the subject is compressed by the compression plate 13. The radiation R is transmitted through the compression plate 13, the breast of the subject, and the imaging table 24 and reaches the radiation detector 15.

The radiation detector 15 detects the radiation R transmitted through a breast of the subject, and generates radiation image data of the breast of the subject based on the detected radiation R. Hereinafter, the radiation image data is simply referred to as a radiation image. A type of the radiation detector 15 is not particularly limited and may be, for example, a so-called indirect conversion type radiation detector that converts the radiation R into light and converts the converted light into a charge, or a direct conversion type radiation detector that directly converts the radiation R into a charge.

The radiation image memory 16 stores the radiation image generated by the radiation detector 15 under the control of the radiography apparatus controller 21. Here, as the radiation image memory 16, for example, recording media such as a flash memory, a hard disk drive (HDD), a solid state drive (SSD), a flexible disk (FD), a magneto-optical disk (MO disk), a magnetic tape (MT), a random access memory (RAM), a compact disc (CD), a digital versatile disc (DVD), a secure digital card (SD card), or a universal serial bus memory (USB memory) can be used.

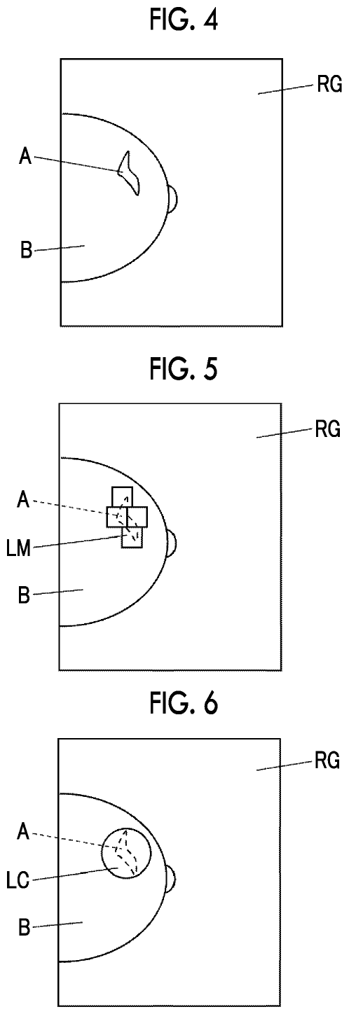

The mammary gland region detection unit 17 analyzes the radiation image of the breast of the subject, thereby detecting a mammary gland region A as shown in FIG. 4. Here, in general, the mammary gland region A in a radiation image RG of a breast B is depicted as a high-brightness region because it is relatively difficult to transmit radiation. Therefore, for example, the mammary gland region detection unit 17 has a brightness threshold value for the radiation image RG, and can detect, as the mammary gland region A, a region having brightness equal to or higher than the brightness threshold value.

The low-resolution image generation unit 18 generates a low-resolution image obtained by reducing a resolution of the mammary gland region A detected by the mammary gland region detection unit 17. The low-resolution image generation unit 18 can generate a low-resolution image LM obtained by performing so-called mosaic processing on the mammary gland region A, as shown in FIG. 5, for example. In addition, the low-resolution image generation unit 18 can generate a circle surrounding the mammary gland region A as a low-resolution image LC, as shown in FIG. 6, for example. The low-resolution image generation unit 18 can generate, as the low-resolution image, an optional image indicating a position of the mammary gland region A and having a shape different from the shape of the mammary gland region A, in addition to the mosaic-like low-resolution image LM and the circular low-resolution image LC.

The projection mapping device 19 performs so-called projection mapping of applying visible light V onto an upper surface 13A of the compression plate 13 to draw the low-resolution image LM generated by the low-resolution image generation unit 18 on the compression plate 13, under the control of the radiography apparatus controller 21. Since the compression plate 13 is transparent, in a case in which the projection mapping device 19 draws the low-resolution image LM on the compression plate 13 that is compressing the breast B of the subject, the low-resolution image LM appears to be superimposed on the compressed breast B.

The projection mapping device 19 can be attached to the radiography apparatus 1, for example, as shown in FIG. 3, and can also be disposed outside the radiography apparatus 1 at a position where the upper surface 13A of the compression plate 13 can be irradiated with the visible light V.

The communication circuit 20 is connected to the console 2 through so-called wired communication or so-called wireless communication, transmits information such as the radiation image RG to the console 2, and receives instruction information for operating each unit of the radiography apparatus 1 from the console 2.

The input device 22 is a device on which a user such as a doctor or a technician performs an input operation. The input device 22 can be provided, for example, as a plurality of switches on the imaging table 24 of the radiography apparatus 1 or the like. The input device 22 may be a mechanical switch or may be a touch panel type switch. In addition, the input device 22 may be a foot switch on which the user performs an input operation with his or her foot.

The radiography apparatus controller 21 controls each unit of the radiography apparatus 1 in accordance with a program and the like recorded in advance.

Although the processor 23 having the mammary gland region detection unit 17, the low-resolution image generation unit 18, and the radiography apparatus controller 21 is configured of a central processing unit (CPU) and a control program for causing the CPU to execute various kinds of processing, the processor 23 may be configured using a field programmable gate array (FPGA), a digital signal processor (DSP), an application specific integrated circuit (ASIC), a graphics processing unit (GPU), and other integrated circuits (ICs) or may be configured by a combination thereof.

In addition, the mammary gland region detection unit 17, the low-resolution image generation unit 18, and the radiography apparatus controller 21 of the processor 23 can be configured to be partially or entirely integrated into one CPU or the like.

The console 2 displays the radiation image RG captured by the radiography apparatus 1 and an ultrasound image captured by the ultrasound diagnostic apparatus 4, which will be described below, in addition to controlling the radiography apparatus 1 and the ultrasound diagnostic apparatus 4 by using imaging instruction information acquired from the RIS or the like.

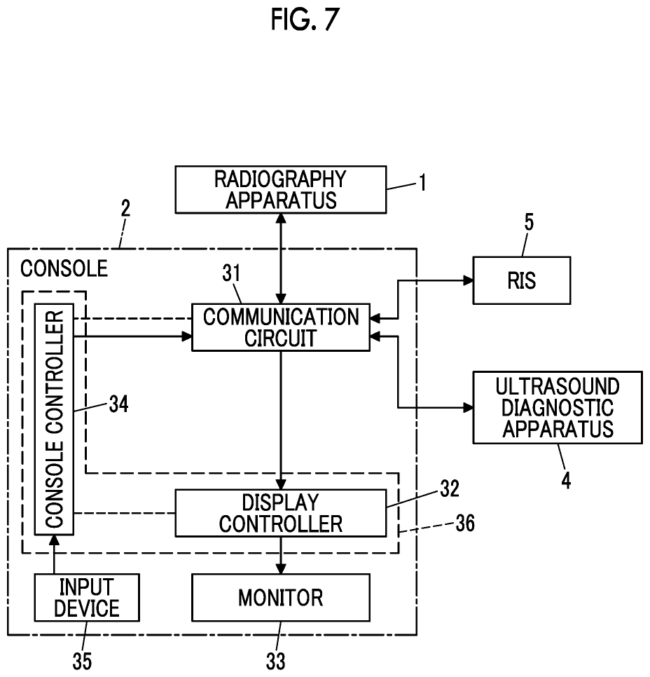

As shown in FIG. 7, the console 2 includes a communication circuit 31 connected to the radiography apparatus 1, the ultrasound diagnostic apparatus 4, and the RIS 5. A display controller 32 and a monitor 33 are sequentially connected to the communication circuit 31. In addition, a console controller 34 is connected to the communication circuit 31 and the display controller 32. An input device 35 is connected to the console controller 34. In addition, the display controller 32 and the console controller 34 constitute a processor 36 for the console 2.

The communication circuit 31 is connected to the radiography apparatus 1, the ultrasound diagnostic apparatus 4, and the RIS 5 through wired communication or wireless communication, and exchanges the radiation image RG or information such as information instruction information with the radiography apparatus 1, the ultrasound diagnostic apparatus 4, and the RIS 5.

The display controller 32 performs predetermined processing on the radiation image RG transmitted from the radiography apparatus 1, the ultrasound image transmitted from the ultrasound diagnostic apparatus 4, and the like to display the images on the monitor 33 under the control of the console controller 34.

The monitor 33 performs various kinds of display under the control of the display controller 32. The monitor 33 can include a display device such as a liquid crystal display (LCD) or an organic electroluminescence (EL) display.

The input device 35 is used for a user to input an instruction or the like related to the capturing of the radiation image RG. The input device 35 can include a mechanical switch, a touch panel, a mouse, or the like.

The console controller 34 controls each unit of the console 2 in accordance with a program and the like recorded in advance.

Although the processor 36 having the display controller 32 and the console controller 34 is configured of a CPU and a control program for causing the CPU to execute various kinds of processing, the processor 36 may be configured using an FPGA, a DSP, an ASIC, a GPU, and other ICs or may be configured by a combination thereof. In addition, the display controller 32 and the console controller 34 of the processor 36 can be configured to be partially or entirely integrated into one CPU or the like.

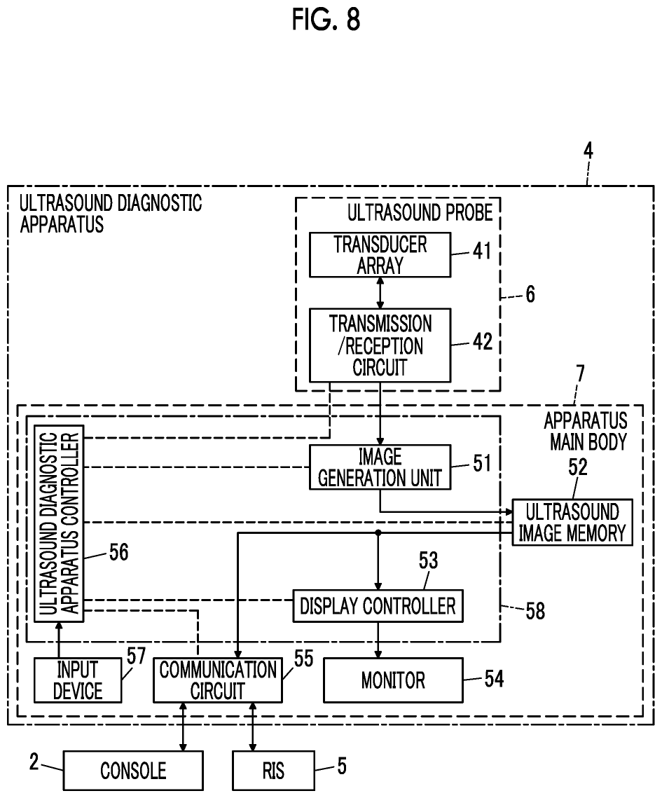

As shown in FIG. 8, the ultrasound diagnostic apparatus 4 comprises an ultrasound probe 6 and an apparatus main body 7 connected to the ultrasound probe 6. The ultrasound probe 6 includes a transducer array 41, and a transmission/reception circuit 42 is connected to the transducer array 41.

The apparatus main body 7 comprises an image generation unit 51 connected to the transmission/reception circuit 42 of the ultrasound probe 6. An ultrasound image memory 52 is connected to the image generation unit 51. A display controller 53 and a monitor 54 are sequentially connected to the ultrasound image memory 52. In addition, a communication circuit 55 is connected to the ultrasound image memory 52. The communication circuit 55 is connected to the console 2 and the RIS 5. In addition, an ultrasound diagnostic apparatus controller 56 is connected to the transmission/reception circuit 42, the image generation unit 51, the ultrasound image memory 52, the display controller 53, and the communication circuit 55. An input device 57 is connected to the ultrasound diagnostic apparatus controller 56.

In addition, the image generation unit 51, the display controller 53, and the ultrasound diagnostic apparatus controller 56 constitute a processor 58 for the ultrasound diagnostic apparatus 4.

The transducer array 41 of the ultrasound probe 6 has a plurality of ultrasound transducers arranged one-dimensionally or two-dimensionally. Each of these ultrasound transducers transmits an ultrasound wave in accordance with a drive signal supplied from the transmission/reception circuit 42 and receives an ultrasound echo from the subject to output a signal based on the ultrasound echo. Each ultrasound transducer is configured by forming electrodes at both ends of a piezoelectric body consisting of, for example, a piezoelectric ceramic represented by lead zirconate titanate (PZT), a polymer piezoelectric element represented by poly vinylidene di fluoride (PVDF), a piezoelectric single crystal represented by lead magnesium niobate-lead titanate (PMN-PT), or the like.

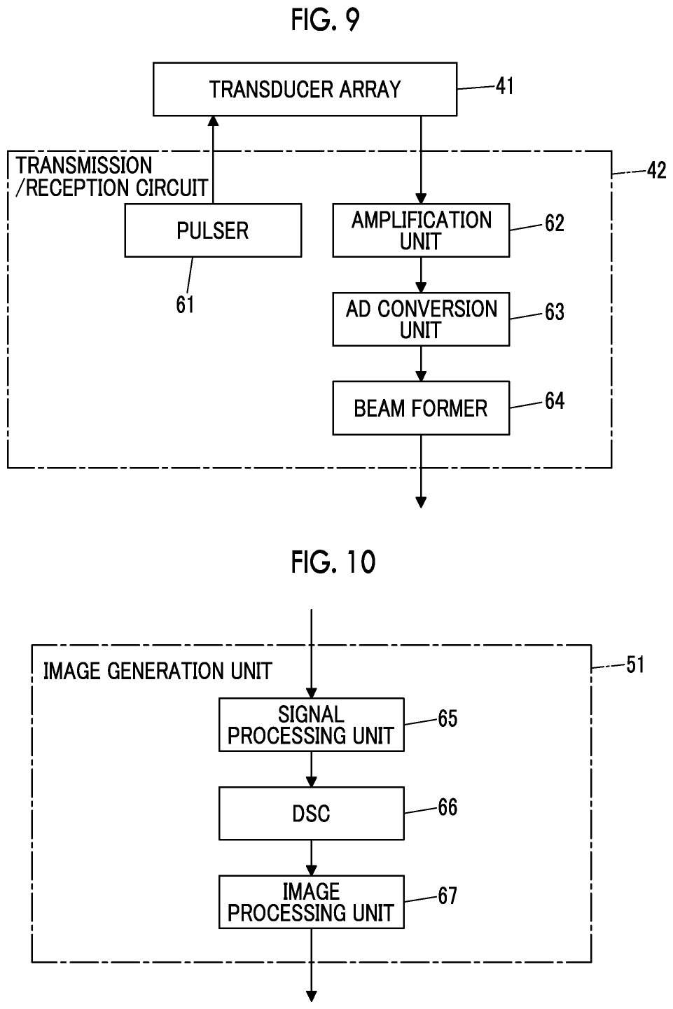

The transmission/reception circuit 42 transmits the ultrasound wave from the transducer array 41 and generates a sound ray signal based on a reception signal acquired by the transducer array 41 under the control of the ultrasound diagnostic apparatus controller 56. As shown in FIG. 9, the transmission/reception circuit 42 includes a pulser 61 connected to the transducer array 41, and an amplification unit 62, an analog to digital (AD) conversion unit 63, and a beam former 64 that are sequentially connected in series to the transducer array 41.

The pulser 61 includes, for example, a plurality of pulse generators, adjusts a delay amount of each drive signal based on a transmission delay pattern selected in accordance with a control signal from the ultrasound diagnostic apparatus controller 56 so that the ultrasound waves transmitted from the plurality of ultrasound transducers of the transducer array 41 form an ultrasound beam, and supplies each drive signal to the plurality of ultrasound transducers. In this way, in a case in which a pulsed or continuous wave-like voltage is applied to the electrodes of the ultrasound transducer of the transducer array 41, the piezoelectric body expands and contracts to generate a pulsed or continuous wave-like ultrasound wave from each of the ultrasound transducers, whereby an ultrasound beam is formed from a combined wave of these ultrasound waves.

The transmitted ultrasound beam is reflected in, for example, a target such as a site of the subject and propagates toward the transducer array 41 of the ultrasound probe 6. The ultrasound echo propagating toward the transducer array 41 in this way is received by each of the ultrasound transducers constituting the transducer array 41. In this case, each of the ultrasound transducers constituting the transducer array 41 expands and contracts by receiving the propagating ultrasound echo to generate a reception signal, which is an electrical signal, and outputs these reception signals to the amplification unit 62.

The amplification unit 62 amplifies the signal input from each of the ultrasound transducers constituting the transducer array 41 and transmits the amplified signal to the AD conversion unit 63. The AD conversion unit 63 converts the signal transmitted from the amplification unit 62 into digital reception data. The beam former 64 performs so-called reception focus processing of applying delays to respective pieces of the reception data received from the AD conversion unit 63 and adding up the results. Through this reception focus processing, a sound ray signal in which each reception data converted by the AD conversion unit 63 is phase-added and the focus of the ultrasound echo is narrowed down is acquired.

As shown in FIG. 10, the image generation unit 51 has a configuration in which a signal processing unit 65, a digital scan converter (DSC) 66, and an image processing unit 67 are sequentially connected in series.

The signal processing unit 65 corrects attenuation by distance of the sound ray signal received from the transmission/reception circuit 42 in accordance with depths of reflection positions of the ultrasound waves using a sound speed value set by the ultrasound diagnostic apparatus controller 56, and then performs envelope detection processing on the sound ray signal to generate a B-mode image signal that is tomographic image information related to tissues inside the subject.

The DSC 66 converts (raster-converts) the B-mode image signal generated in the signal processing unit 65 into an image signal according to a normal television signal scanning method.

The image processing unit 67 performs various kinds of necessary image processing such as gradation processing on the B-mode image signal input from the DSC 66, and then sends out the B-mode image signal to the display controller 53 and the communication circuit 55. Hereinafter, the B-mode image signal that has been subjected to image processing by the image processing unit 67 is referred to as an ultrasound image.

The ultrasound image memory 52 stores the ultrasound image generated by the image generation unit 51. As the ultrasound image memory 52, for example, recording media such as a flash memory, an HDD, an SSD, an FD, an MO disk, an MT, a RAM, a CD, a DVD, an SD card, and a USB memory can be used.

The display controller 53 performs predetermined processing on the ultrasound image or the like generated by the image generation unit 51 and displays the ultrasound image or the like on the monitor 54 under the control of the ultrasound diagnostic apparatus controller 56.

The monitor 54 performs various kinds of display under the control of the display controller 53. The monitor 54 can include, for example, a display device such as an LCD or an organic EL display.

The communication circuit 55 is connected to the console 2 and the RIS 5 through wired communication or wireless communication, and exchanges the ultrasound image or information such as information instruction information with the console 2 and the RIS 5.

The ultrasound diagnostic apparatus controller 56 controls each unit of the apparatus main body 7 and the ultrasound probe 6 in accordance with a program recorded in advance, or the like.

The input device 57 receives an input operation of the user and sends out input information to the ultrasound diagnostic apparatus controller 56. The input device 57 is configured of, for example, a device for the user to perform the input operation, such as a keyboard, a mouse, a trackball, a touch pad, and a touch panel.

Although the processor 58 having the image generation unit 51, the display controller 53, and the ultrasound diagnostic apparatus controller 56 is configured of a CPU and a control program for causing the CPU to execute various kinds of processing, the processor 58 may be configured using an FPGA, a DSP, an ASIC, a GPU, and other ICs or may be configured by a combination thereof. In addition, the image generation unit 51, the display controller 53, and the ultrasound diagnostic apparatus controller 56 of the processor 58 can also be configured to be partially or entirely integrated into one CPU or the like.

The RIS 5 is a system that manages an examination using the radiography system 3 and the ultrasound diagnostic apparatus 4 for each of a plurality of subjects, together with examination results, and can be configured of, for example, a server apparatus or a computer (not shown). The user can access the RIS 5 to make a reservation for an examination, recording of an examination result, viewing of an examination result, and the like for each subject.

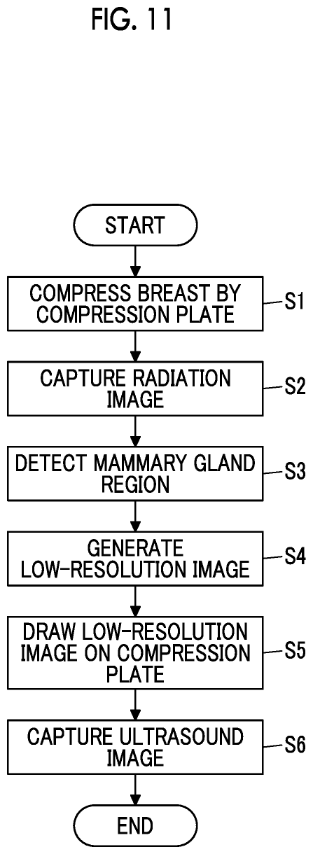

Next, an example of an operation of the medical image diagnostic system according to Embodiment 1 will be described with reference to a flowchart of FIG. 11.

First, in step S1, the breast B of the subject is compressed by the compression plate 13. In this case, in a state where the breast B of the subject is disposed on the imaging surface 24A of the imaging table 24, for example, the radiography apparatus controller 21 can operate the compression plate driving unit 14 based on the instruction information input by the user via the input device 22 of the radiography apparatus 1 to compress the breast B by the compression plate 13 by moving the compression plate 13.

Next, in step S2, the radiation image RG of the breast B compressed by the compression plate 13 is captured. In this case, the radiation source moving unit 12 adjusts the inclination angle of the radiation source 11 based on the input operation of the user via the input device 22, for example, and then the breast B compressed by the compression plate 13 is irradiated with the radiation R from the radiation source 11. The radiation R emitted here is transmitted through the compression plate 13, the breast B of the subject, and the imaging table 24 and reaches the radiation detector 15. The radiation detector 15 detects the radiation R, and generates the radiation image RG of the breast B of the subject, for example, as shown in FIG. 4, based on the detected radiation R. The radiation image RG generated in this manner is stored in the radiation image memory 16 as image data.

In step S3, the mammary gland region detection unit 17 reads out and analyzes the radiation image RG that is generated and stored in the radiation image memory 16 in step S2, thereby detecting the mammary gland region A in the breast B of the subject. In general, the mammary gland region A in the radiation image RG of the breast B is depicted as a region having high brightness, so that the mammary gland region detection unit 17 can detect, as the mammary gland region A, a region having brightness equal to or higher than a predetermined brightness threshold value, for example.

In step S4, the low-resolution image generation unit 18 generates a low-resolution image by reducing a resolution of the image of the mammary gland region A detected in step S3. The low-resolution image generation unit 18 can generate the mosaic-like low-resolution image LM as shown in FIG. 5, for example, by performing mosaic processing on the image of the mammary gland region A. In addition, the low-resolution image generation unit 18 can also generate the circular low-resolution image LC indicating a position of the mammary gland region A, for example. In the following description of the flowchart, an example in which the mosaic-like low-resolution image LM is generated will be described.

In step S5, the radiography apparatus controller 21 causes the projection mapping device 19 to draw the low-resolution image LM generated in step S4 on the upper surface 13A of the compression plate 13. Since the compression plate 13 is transparent, in a case in which the projection mapping device 19 draws the low-resolution image LM on the compression plate 13 that is compressing the breast B of the subject, the low-resolution image LM appears to be superimposed on the compressed breast B.

The user can accurately understand the position of the mammary gland region A in the compressed breast B by confirming the low-resolution image LM drawn on the compression plate 13 in this manner. In addition, some subjects may avoid confirming the detailed shape of their own mammary gland region A. However, since the low-resolution image LM is depicted on the compression plate 13, even in a case in which the subject unintentionally confirms the low-resolution image LM depicted on the compression plate 13, it is possible to prevent the subject from understanding the detailed shape of the mammary gland region A.

Finally, in step S6, an ultrasound image of the mammary gland region A is captured in a state where the distal end of the ultrasound probe 6 is disposed by the user at the position of the low-resolution image LM depicted on the compression plate 13 in step S5, that is, at the position of the mammary gland region A. The ultrasound image generated here is stored in the ultrasound image memory 52 of the ultrasound diagnostic apparatus 4, displayed on the monitor 54, and is also transmitted to the console 2 and the RIS 5 to be viewed or stored.

In a case in which the ultrasound image is captured, the user can dispose the distal end of the ultrasound probe 6 on the mammary gland region A while confirming the low-resolution image LM on the compression plate 13, so that the mammary gland region A can be easily and accurately examined.

In a case in which the processing of step S6 is completed in this manner, the operation of the medical image diagnostic system in accordance with the flowchart of FIG. 11 is completed.

As described above, with the medical image diagnostic system according to Embodiment 1, the low-resolution image generation unit 18 generates the low-resolution image LM obtained by reducing the resolution of the mammary gland region A detected by the mammary gland region detection unit 17, and the image drawing unit draws the low-resolution image LM on the compression plate 13 by using the projection mapping device 19. Therefore, it is possible for the user to easily and accurately examine the mammary gland region A of the subject while preventing the subject from confirming the detailed shape of the mammary gland region A.

Although the description has been made in which the mammary gland region detection unit 17 and the low-resolution image generation unit 18 are provided in the radiography apparatus 1, the mammary gland region detection unit 17 and the low-resolution image generation unit 18 can also be provided in the ultrasound diagnostic apparatus 4, for example. In this case, the radiation image RG of the breast B is transmitted from the radiography apparatus 1 to the ultrasound diagnostic apparatus 4 via the console 2, the mammary gland region A reflected in the radiation image RG is detected by the mammary gland region detection unit 17 of the ultrasound diagnostic apparatus 4, and the low-resolution image LM is generated by the low-resolution image generation unit 18. The low-resolution image LM generated in the ultrasound diagnostic apparatus 4 in this manner is transmitted from the ultrasound diagnostic apparatus 4 to the radiography apparatus 1 via the console 2. In the radiography apparatus 1, the low-resolution image LM transmitted from the ultrasound diagnostic apparatus 4 is sent out to the projection mapping device 19. Thereafter, the low-resolution image LM is depicted on the compression plate 13 by the projection mapping device 19 under the control of the radiography apparatus controller 21.

Similarly, the mammary gland region detection unit 17 and the low-resolution image generation unit 18 can be provided in the console 2.

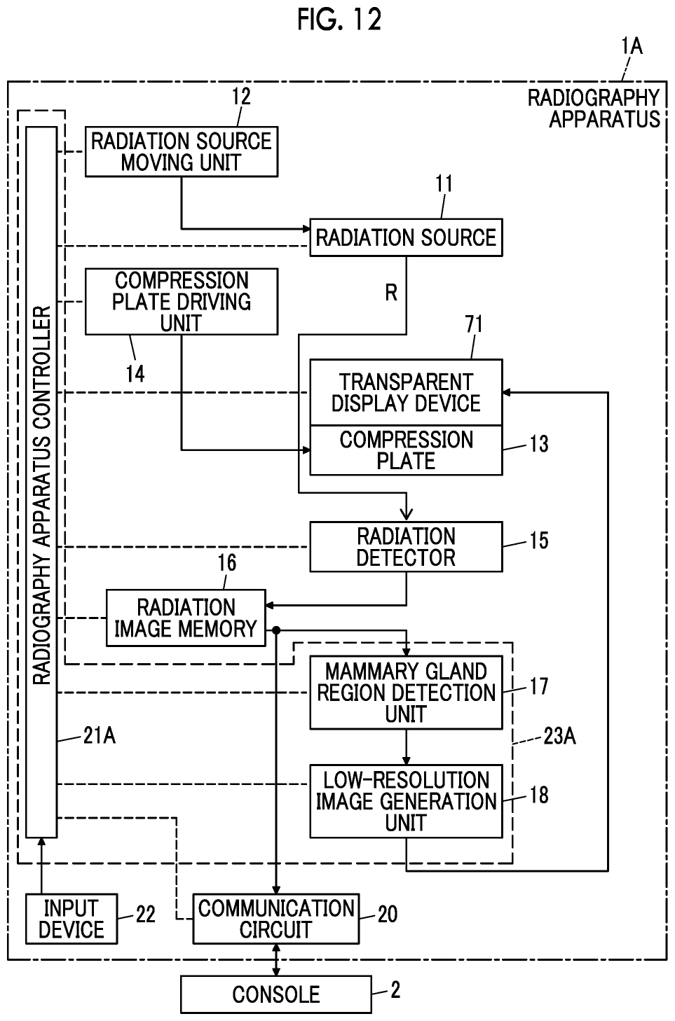

In addition, although a case in which an image depiction unit that depicts the low-resolution image LM on the compression plate 13 includes the projection mapping device 19 has been described, the image depiction unit can include any configuration that can draw an image on the compression plate 13 instead of the projection mapping device 19. The image depiction unit can also include a so-called transparent display device 71, for example, as shown in FIG. 12.

Here, a radiography apparatus 1A of FIG. 12 includes the transparent display device 71 instead of the projection mapping device 19 in the radiography apparatus 1 of FIG. 2, and includes a radiography apparatus controller 21A instead of the radiography apparatus controller 21. In addition, the mammary gland region detection unit 17, the low-resolution image generation unit 18, and the radiography apparatus controller 21A constitute a processor 23A.

The transparent display device 71 is a device that has a transparent display screen and can display an image on the display screen. The transparent display device 71 is disposed to be superimposed on the compression plate 13, and displays the low-resolution image LM generated by the low-resolution image generation unit 18 under the control of the radiography apparatus controller 21A. Since the display screen of the transparent display device 71 and the compression plate 13 are transparent, in a case in which the transparent display device 71 displays the low-resolution image LM, the low-resolution image LM appears to be superimposed on the compressed breast B.

Therefore, even in a case in which the radiography apparatus 1A includes the transparent display device 71, it is possible for the user to easily and accurately examine the mammary gland region A of the subject while preventing the subject from confirming the detailed shape of the mammary gland region A, as in the case in which the radiography apparatus 1 includes the projection mapping device 19.

In addition, although a case in which the transmission/reception circuit 42 is provided in the ultrasound probe 6 in the ultrasound diagnostic apparatus 4 has been described, the transmission/reception circuit 42 may be provided in the apparatus main body 7.

In addition, although a case in which the image generation unit 51 is provided in the apparatus main body 7 has been described, the image generation unit 51 may be provided in the ultrasound probe 6.

In addition, the apparatus main body 7 may be a so-called stationary type, a portable type that is easy to carry, or a so-called handheld type that is composed of, for example, a smartphone or a tablet type computer. As described above, a type of the device that constitutes the apparatus main body 7 is not particularly limited.

### Embodiment 2

In Embodiment 1, a case in which the low-resolution image LM is drawn on the compression plate 13 has been described, but, by further displaying a position of the mammary gland region A before being reduced in resolution on the compression plate 13, the user can more clearly understand the position of the mammary gland region A.

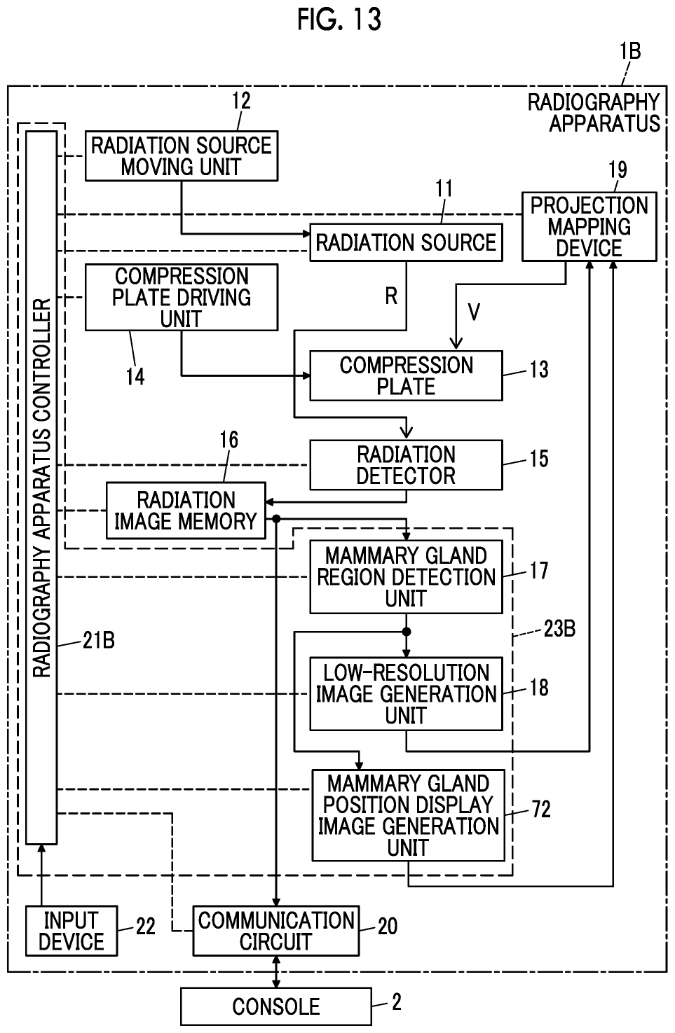

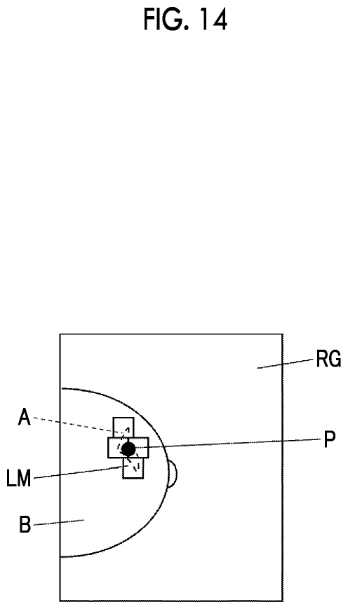

The medical image diagnostic system according to Embodiment 2 includes, for example, a radiography apparatus 1B shown in FIG. 13. The radiography apparatus 1B further comprises a mammary gland position display image generation unit 72, and comprises a radiography apparatus controller 21B instead of the radiography apparatus controller 21 in the radiography apparatus 1 in Embodiment 1 shown in FIG. 2. In the radiography apparatus 1B, the mammary gland position display image generation unit 72 is connected to the mammary gland region detection unit 17. The mammary gland position display image generation unit 72 is connected to the projection mapping device 19 and the radiography apparatus controller 21B. In addition, the mammary gland region detection unit 17, the low-resolution image generation unit 18, the mammary gland position display image generation unit 72, and the radiography apparatus controller 21B constitute a processor 23B for the radiography apparatus 1B.

The mammary gland position display image generation unit 72 generates a mammary gland position display image indicating the position of the mammary gland region A based on the mammary gland region A detected by the mammary gland region detection unit 17. For example, the mammary gland position display image generation unit 72 can, for example, calculate a position of the centroid of the mammary gland region A detected by the mammary gland region detection unit 17, and generate a centroid image P as shown in FIG. 14, for example, which represents the calculated position of the centroid, as the mammary gland position display image. The mammary gland position display image is not limited to the centroid image P as long as the image indicates the position of the mammary gland region A and does not represent the detailed shape of the mammary gland region A, and may be, for example, an image having a shape such as a circular, elliptical, or any polygonal shape that indicates the position of the mammary gland region A.

The image drawing unit uses the projection mapping device 19 to draw the mammary gland position display image generated by the mammary gland position display image generation unit 72 to be superimposed on the low-resolution image LM drawn on the compression plate 13.

As described above, with the medical image diagnostic system of Embodiment 2, the mammary gland position display image generation unit 72 generates the mammary gland position display image indicating the position of the mammary gland region A, and the image drawing unit draws the mammary gland position display image to be superimposed on the low-resolution image LM drawn on the compression plate 13, so that the user can more accurately dispose the distal end of the ultrasound probe 6 at the position of the mammary gland region A by conforming the mammary gland position display image together with the low-resolution image LM.

Although a case in which the mammary gland position display image generation unit 72 is provided in the radiography apparatus 1B has been described, the mammary gland position display image generation unit 72 can be provided in the console 2 or can be provided in the ultrasound diagnostic apparatus 4.

### Embodiment 3

In Embodiment 1 and Embodiment 2, a case in which the low-resolution image LM is displayed on the compression plate 13 has been described, but, in order for the user to understand the mammary gland region A that has been scanned and the mammary gland region A that is not yet scanned and to perform the examination more smoothly, a display aspect of the low-resolution image LM corresponding to the mammary gland region A that has been scanned can be changed before and after the scanning.

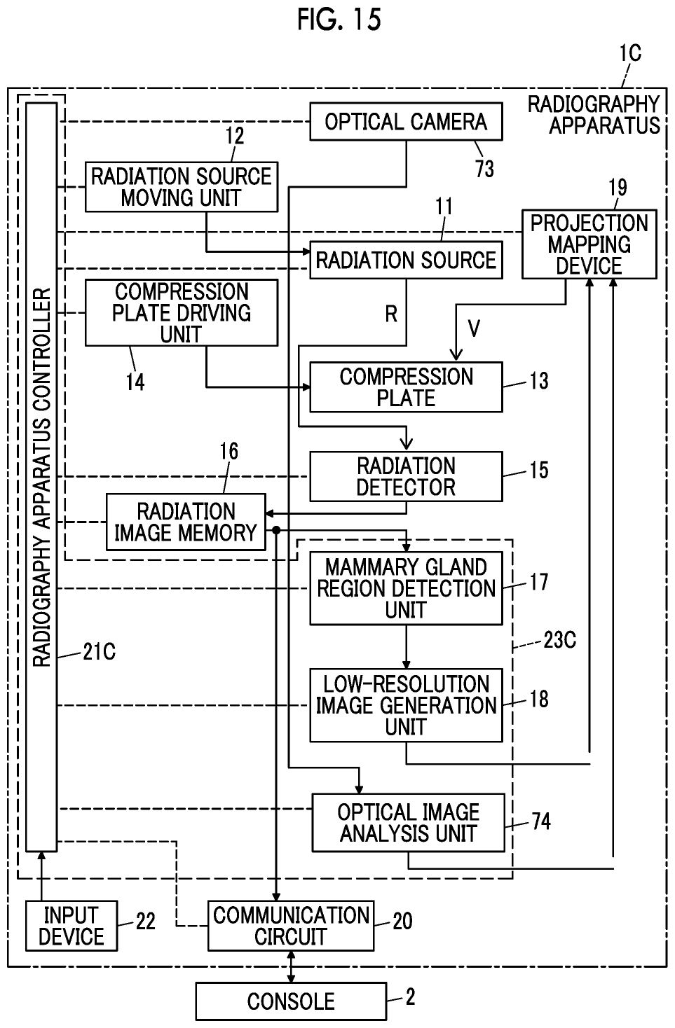

FIG. 15 shows a configuration of a radiography apparatus 1C in the medical image diagnostic system of Embodiment 3. The radiography apparatus 1C further comprises an optical camera 73 and an optical image analysis unit 74, and comprises a radiography apparatus controller 21C instead of the radiography apparatus controller 21 in the radiography apparatus 1 in Embodiment 1 shown in FIG. 2.

In the radiography apparatus 1C, the optical camera 73 is connected to the radiography apparatus controller 21C. The optical image analysis unit 74 is connected to the optical camera 73. The optical image analysis unit 74 is connected to the projection mapping device 19 and the radiography apparatus controller 21C. In addition, the optical camera 73 and the optical image analysis unit 74 constitute a probe-disposed image recognition unit.

The optical camera 73 is attached to, for example, the arm part 25 of the radiography apparatus 1C, and captures an optical image of the breast B of the subject compressed by the compression plate 13. The optical camera 73 includes, for example, an image sensor such as a so-called charge coupled device (CCD) image sensor or a so-called a complementary metal-oxide-semiconductor (CMOS) image sensor.

The optical image analysis unit 74 detects the low-resolution image LM depicted on the compression plate 13 and the ultrasound probe 6 by analyzing the optical image captured by the optical camera 73, and recognizes the low-resolution image LM that is drawn on the compression plate 13 by the image drawing unit and on which the distal end of the ultrasound probe 6 is disposed. The optical image analysis unit 74 can detect the low-resolution image LM and the ultrasound probe 6 reflected in the optical image by, for example, a so-called template matching method. In addition, the optical image analysis unit 74 can detect the low-resolution image LM and the ultrasound probe 6 reflected in the optical image by using a machine learning model that has been trained using a plurality of low-resolution images LM and a plurality of optical images of the ultrasound probe 6 as training data, and recognize the low-resolution image LM on which the distal end of the ultrasound probe 6 is disposed.

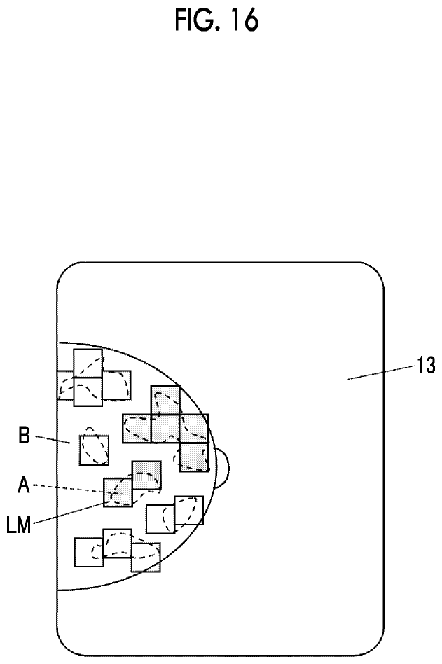

The image drawing unit can use the projection mapping device 19 to change the display aspect on the compression plate 13 of the low-resolution image LM recognized by the optical image analysis unit 74 as shown in FIG. 16, for example. FIG. 16 shows an example in which six low-resolution images LM corresponding to six mammary gland regions A are drawn on the compression plate 13, and a display color of two low-resolution images LM among the six low-resolution images LM is changed. The image drawing unit can perform any change of the display aspect such as changing a thickness of an outline of the low-resolution image LM or blinking the low-resolution image LM in addition to changing the display color of the low-resolution image LM.

As described above, with the medical image diagnostic system of Embodiment 3, the probe-disposed image recognition unit recognizes the low-resolution image LM that is drawn on the compression plate 13 and on which the distal end of the ultrasound probe 6 is disposed, and the image drawing unit changes the display aspect on the compression plate 13 of the low-resolution image LM recognized by the probe-disposed image recognition unit, so that the user can easily understand the mammary gland region A that has been scanned and the mammary gland region A that is not yet scanned and can perform the examination more smoothly.

Although a case in which the optical camera 73 is provided in the radiography apparatus 1C has been described, the optical camera 73 can also be disposed outside the radiography apparatus 1C as long as the optical camera 73 is in a position where the optical images of the compression plate 13 and the ultrasound probe 6 disposed on the compression plate 13 can be clearly captured.

Although a case in which the optical image analysis unit 74 is provided in the radiography apparatus 1C has been described, the optical image analysis unit 74 may be provided in, for example, the console 2 or the ultrasound diagnostic apparatus 4.

In addition, a case in which the optical camera 73 and the optical image analysis unit 74 constitute the probe-disposed image recognition unit has been described, the configuration of the probe-disposed image recognition unit is not particularly limited to this. For example, the probe-disposed image recognition unit can also recognize the low-resolution image LM that is drawn on the compression plate 13 by the image drawing unit and on which the distal end of the ultrasound probe 6 is disposed by analyzing information obtained by a position sensor that detects the position of the ultrasound probe 6.

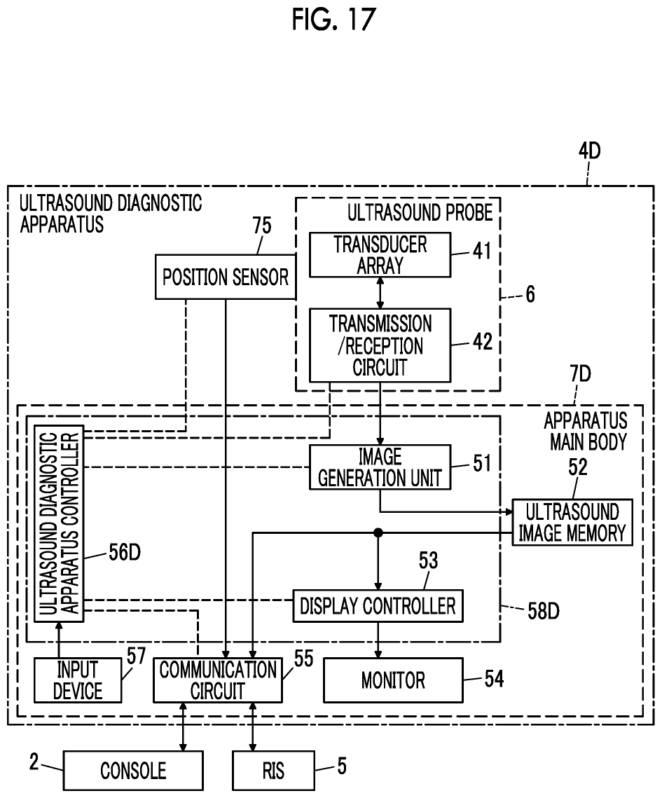

For example, FIG. 17 shows a configuration of an ultrasound diagnostic apparatus 4D in a modification example of Embodiment 3. The ultrasound diagnostic apparatus 4D further comprises a position sensor 75 attached to the ultrasound probe 6, and comprises an apparatus main body 7D instead of the apparatus main body 7 in the ultrasound diagnostic apparatus 4 in Embodiment 4 shown in FIG. 8. The apparatus main body 7D comprises an ultrasound diagnostic apparatus controller 56D instead of the ultrasound diagnostic apparatus controller 56 in the apparatus main body 7. In the ultrasound diagnostic apparatus 4D, the position sensor 75 is connected to the communication circuit 55 and the ultrasound diagnostic apparatus controller 56D. In addition, the image generation unit 51, the display controller 53, and the ultrasound diagnostic apparatus controller 56D constitute a processor 58D for the apparatus main body 7D.

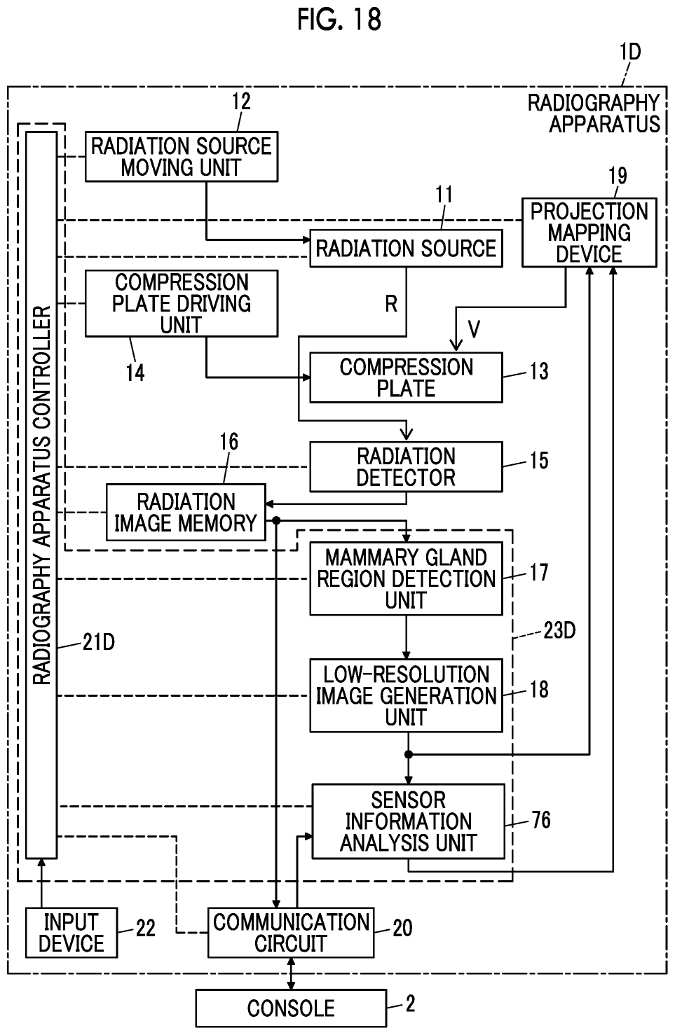

The position sensor 75 includes, for example, a sensor device such as an acceleration sensor, a gyro sensor, a magnetic sensor, or a global positioning system (GPS) sensor, and detects the position of the distal end of the ultrasound probe 6. A detection signal of the position sensor 75 is transmitted from the communication circuit 55 to, for example, the radiography apparatus 1D shown in FIG. 18 via the console 2.

The radiography apparatus 1D further comprises a sensor information analysis unit 76, and comprises a radiography apparatus controller 21D instead of the radiography apparatus controller 21 in the radiography apparatus 1 in Embodiment 1 shown in FIG. 2. In the radiography apparatus 1D, the sensor information analysis unit 76 is connected to the low-resolution image generation unit 18 and the communication circuit 20. The sensor information analysis unit 76 is connected to the projection mapping device 19 and the radiography apparatus controller 21D. The position sensor 75 of the ultrasound diagnostic apparatus 4D and the sensor information analysis unit 76 of the radiography apparatus 1D constitute a probe-disposed image recognition unit. In addition, the mammary gland region detection unit 17, the low-resolution image generation unit 18, the sensor information analysis unit 76, and the radiography apparatus controller 21D constitute a processor 23D for the radiography apparatus 1D.

The sensor information analysis unit 76 receives information regarding the disposition position of the low-resolution image LM from the low-resolution image generation unit 18, and receives the detection signal regarding the position of the distal end of the ultrasound probe 6 detected by the position sensor 75 and sent out from the communication circuit 20. The sensor information analysis unit 76 can recognize the low-resolution image LM that is drawn on the compression plate 13 by the image drawing unit and on which the distal end of the ultrasound probe 6 is disposed, based on the information regarding the disposition position of the low-resolution image LM and the detection signal regarding the position of the distal end of the ultrasound probe 6.

The sensor information analysis unit 76 may be provided in the console 2 or may be provided in the ultrasound diagnostic apparatus 4D instead of the radiography apparatus 1D.

### Embodiment 4

In a case in which a plurality of mammary gland regions A are detected in the breast B of the subject, the medical image diagnostic system can also notify of a proportion of the mammary gland region A that has been scanned, for example, in order for the user to easily understand a progress status of the examination.

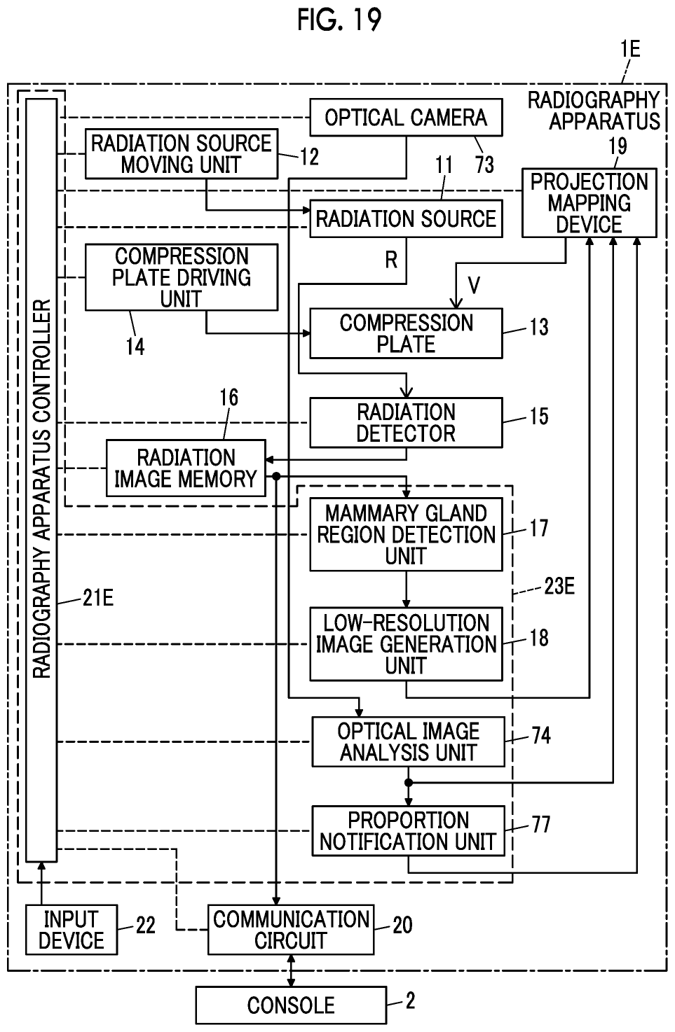

FIG. 19 shows a configuration of a radiography apparatus 1E in the medical image diagnostic system of Embodiment 4. The radiography apparatus 1E further comprises a proportion notification unit 77, and comprises a radiography apparatus controller 21E instead of the radiography apparatus controller 21D in the radiography apparatus 1C in Embodiment 3 shown in FIG. 15.

In the radiography apparatus 1E, the proportion notification unit 77 is connected to the optical image analysis unit 74. The proportion notification unit 77 is connected to the projection mapping device 19 and the radiography apparatus controller 21E. In addition, the mammary gland region detection unit 17, the low-resolution image generation unit 18, the optical image analysis unit 74, the proportion notification unit 77, and the radiography apparatus controller 21E constitute a processor 23E for the radiography apparatus 1E.

In Embodiment 4, the mammary gland region detection unit 17 detects a plurality of mammary gland regions A in the radiation image RG of the breast B of the subject.

The low-resolution image generation unit 18 generates a plurality of low-resolution images LM obtained by reducing the resolutions of the plurality of mammary gland regions A detected by the mammary gland region detection unit 17 as shown in FIG. 16, for example.

The image drawing unit uses the projection mapping device 19 to draw the plurality of low-resolution images LM generated by the low-resolution image generation unit 18 on the compression plate 13.

The proportion notification unit 77 calculates a proportion of the low-resolution image LM recognized by the probe-disposed image recognition unit, more specifically, by the optical image analysis unit 74 from a start of the examination using the ultrasound probe 6 to the present, to the plurality of low-resolution images LM drawn on the compression plate 13, and notifies of the proportion. The proportion notification unit 77 can determine the start of the examination using the ultrasound probe 6 by receiving, for example, a signal indicating that the ultrasound examination has started, which is transmitted from the ultrasound diagnostic apparatus 4D to the radiography apparatus 1E, via the radiography apparatus controller 21E.

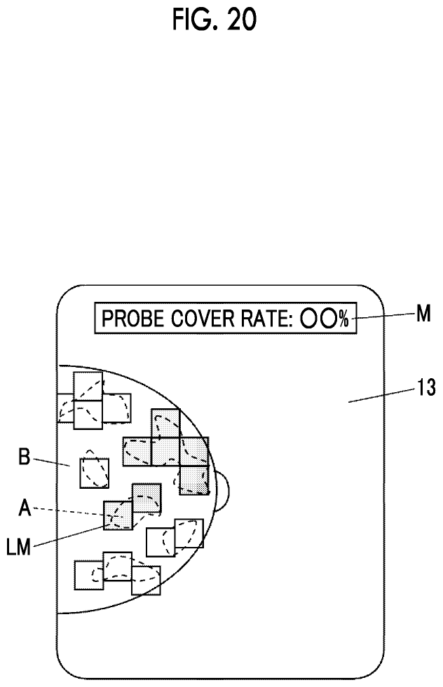

The proportion notification unit 77 can notify of the proportion by drawing the calculated proportion as a message M on the compression plate 13 by using the projection mapping device 19 as shown in FIG. 20, for example. The user can easily understand the progress status of the examination and can smoothly proceed with the examination by confirming the message M.

As described above, with the medical image diagnostic system of Embodiment 4, the proportion notification unit 77 calculates the proportion of the low-resolution image LM recognized by the probe-disposed image recognition unit from the start of the examination using the ultrasound probe 6 to the present, to the plurality of low-resolution images LM drawn on the compression plate 13, and notifies of the proportion, so that the user can easily understand the progress status of the examination and can smoothly proceed with the examination.

The proportion notification unit 77 may be provided in the console 2 or may be provided in the ultrasound diagnostic apparatus 4D instead of being provided in the radiography apparatus 1E.

In addition, a case in which the proportion notification unit 77 draws the message M on the compression plate 13 to notify of the proportion of the mammary gland region A that has been scanned has been described, but a method of the notification is not limited to this. For example, in a case in which the medical image diagnostic system has a speaker (not shown), the proportion notification unit 77 can also notify of the proportion of the mammary gland region A that has been scanned with voice via the speaker. In addition, the proportion notification unit 77 can also notify of the proportion of the mammary gland region A that has been scanned by displaying the message M on the monitor 33 of the console 2 or on the monitor 54 of the ultrasound diagnostic apparatus 4D.

### EXPLANATION OF REFERENCES

1, 1A, 1B, 1C, 1D, 1E: radiography apparatus
2: console
3: radiography system
4: ultrasound diagnostic apparatus
5: RIS
6: ultrasound probe
7: apparatus main body
11: radiation source
12: radiation source moving unit
13: compression plate
14: compression plate driving unit
15: radiation detector
16: radiation image memory
17: mammary gland region detection unit
18: low-resolution image generation unit
19: projection mapping device
20, 31, 55: communication circuit
21, 21A, 21B, 21C, 21D, 21E: radiography apparatus controller
22, 35, 57: input device
23, 23A, 23B, 23C, 23D, 23E, 36, 58: processor
24: imaging table
24A: imaging surface
25: arm part
26: base
27: shaft part
32, 53: display controller
33, 54: monitor
34: console controller
41: transducer array
42: transmission/reception circuit
51: image generation unit
52: ultrasound image memory
56: ultrasound diagnostic apparatus controller
61: pulser
62: amplification unit
63: AD conversion unit
64: beam former
65: signal processing unit
66: DSC
67: image processing unit
71: transparent display device
72: mammary gland position display image generation unit
73: optical camera
74: optical image analysis unit
75: position sensor
76: sensor information analysis unit
77: proportion notification unit
A: mammary gland region
B: breast
LC, LM: low-resolution image
M: message
P: centroid image
R: radiation
RG: radiation image
V: visible light

What is claimed is:

1. A medical image diagnostic system comprising:
a compression plate that compresses a breast of a subject;
an ultrasound probe;
a processor configured to
detect a mammary gland region in a radiation image of the breast compressed by the compression plate,
generate a low-resolution image obtained by reducing a resolution of the mammary gland region in the radiation image of the breast; and
an image drawing device configured to draw the low-resolution image on the compression plate,
wherein the mammary gland region of the breast compressed by the compression plate is scanned with the ultrasound probe based on the low-resolution image drawn on the compression plate.

2. The medical image diagnostic system according to claim 1, further comprising:
a probe-disposed image recognition device configured to recognize the low-resolution image that is drawn on the compression plate by the image drawing device and on which a distal end of the ultrasound probe is disposed.

3. The medical image diagnostic system according to claim 2,
wherein the image drawing device configured to change a display aspect on the compression plate of the low-resolution image recognized by the probe-disposed image recognition device.

4. The medical image diagnostic system according to claim 2,
wherein the processor is configured to
detect a plurality of the mammary gland regions in the radiation image of the breast, and
generate a plurality of the low-resolution images obtained by reducing resolutions of the plurality of mammary gland regions in the radiation image of the breast,
the image drawing device configured to draw the plurality of low-resolution images on the compression plate, and
the processor is configured to
calculate a proportion of the low-resolution image recognized by the probe-disposed image recognition device from a start of an examination using the ultrasound probe to the present, to the plurality of low-resolution images drawn on the compression plate, and
notify of the proportion.

5. The medical image diagnostic system according to claim 3,
wherein the processor is configured to
detect a plurality of the mammary gland regions in the radiation image of the breast, and
generate a plurality of the low-resolution images obtained by reducing resolutions of the plurality of mammary gland regions in the radiation image of the breast,
the image drawing device configured to draw the plurality of low-resolution images on the compression plate, and
the processor is configured to
calculate a proportion of the low-resolution image recognized by the probe-disposed image recognition device from a start of an examination using the ultrasound probe to the present, to the plurality of low-resolution images drawn on the compression plate, and
notify of the proportion.

6. The medical image diagnostic system according to claim 1,
wherein the processor is configured to generate a mammary gland position display image indicating a position of the mammary gland region based on the mammary gland region detected in the radiation image of the breast, and
the image drawing device is configured to draw the mammary gland position display image generated by the mammary gland position display image generation unit to be superimposed on the low-resolution image drawn on the compression plate.

7. The medical image diagnostic system according to claim 2,
wherein the processor is configured to generate a mammary gland position display image indicating a position of the mammary gland region based on the mammary gland region detected in the radiation image of the breast, and
the image drawing device is configured to draw the mammary gland position display image generated by the mammary gland position display image generation unit to be superimposed on the low-resolution image drawn on the compression plate.

8. The medical image diagnostic system according to claim 3,
wherein the processor is configured to generate a mammary gland position display image indicating a position of the mammary gland region based on the mammary gland region detected in the radiation image of the breast, and
the image drawing device is configured to draw the mammary gland position display image generated by the mammary gland position display image generation unit to be superimposed on the low-resolution image drawn on the compression plate.

9. The medical image diagnostic system according to claim 4,
wherein the processor is configured to generate a mammary gland position display image indicating a position of the mammary gland region based on the mammary gland region detected in the radiation image of the breast, and
the image drawing device is configured to draw the mammary gland position display image generated by the mammary gland position display image generation unit to be superimposed on the low-resolution image drawn on the compression plate.

10. The medical image diagnostic system according to claim 1,
wherein the image drawing device includes a projection mapping device configured to draw the low-resolution image on the compression plate by projection mapping.

11. The medical image diagnostic system according to claim 2,
wherein the image drawing device includes a projection mapping device configured to draw the low-resolution image on the compression plate by projection mapping.

12. The medical image diagnostic system according to claim 3,
wherein the image drawing device includes a projection mapping device configured to draw the low-resolution image on the compression plate by projection mapping.

13. The medical image diagnostic system according to claim 4,
wherein the image drawing device includes a projection mapping device configured to draw the low-resolution image on the compression plate by projection mapping.

14. The medical image diagnostic system according to claim 6,
wherein the image drawing device includes a projection mapping device configured to draw the low-resolution image on the compression plate by projection mapping.

15. The medical image diagnostic system according to claim 1,
wherein the image drawing device includes a transparent display device disposed on the compression plate configured to draw the low-resolution image.

16. The medical image diagnostic system according to claim 2,
wherein the image drawing device includes a transparent display device disposed on the compression plate configured to draw the low-resolution image.

17. The medical image diagnostic system according to claim 3,
wherein the image drawing device includes a transparent display device disposed on the compression plate configured to draw the low-resolution image.

18. The medical image diagnostic system according to claim 4,
wherein the image drawing device includes a transparent display device disposed on the compression plate configured to draw the low-resolution image.

19. The medical image diagnostic system according to claim 6,
wherein the image drawing device includes a transparent display device disposed on the compression plate configured to draw the low-resolution image.

20. A method of controlling a medical image diagnostic system, the method comprising:
detecting a mammary gland region in a radiation image of a breast of a subject compressed by a compression plate;
generating a low-resolution image obtained by reducing a resolution of the detected mammary gland region; and
drawing the generated low-resolution image on the compression plate,
wherein the mammary gland region of the breast compressed by the compression plate is scanned with an ultrasound probe based on the low-resolution image drawn on the compression plate.
